(19) United States
(12) Patent Application Publication
(10) Pub. No.: US 2026/0140061 A1
(43) Pub. Date: May 21, 2026

(54) SCANNING DEVICE

(71) Applicant: Ventana Medical Systems, Inc., Tuscon, AZ (US)

(72) Inventor: William Hawes, Cambridge (GB)

(21) Appl. No.: 19/501,586

(22) PCT Filed: Jun. 21, 2024
(86) PCT No.: PCT/IB2024/056069

§ 371 Date: Jan. 12, 2026

(30) Foreign Application Priority Data
Jun. 22, 2023 (GB) 2309414.7

(51) Int. Cl. G01N 21/84; G01N 33/483

(52) U.S. Cl. CPC G01N 21/84; G01N 33/4833; G01N 2201/103

(57) ABSTRACT

A scanning device for scanning a target area of a sample, the scanning device comprising: a transportation system arranged to support the sample and move the target area of the sample between a first location and a second location; a casing con-figured to support the transportation system, the casing mechanically coupled to the transportation system such that movement of the transportation system induces oscillatory motion in the casing; a controller configured to: accelerate the transportation system in a first direction, from the first location to the second location, causing a reaction force in the second direction and initial oscillatory motion of the casing in the second direction; and decelerate the transportation system in the first direction to bring the transportation system to a rest when the sample is in the second location, producing a reaction force on the casing in the first direction which balances oscillatory movement of the casing in the second direction, to bring the casing to rest.

### FIELD OF THE INVENTION

The present invention relates to a scanning device for scanning and imaging material, for example biological material, generally in the form of slide specimens such as human tissue specimens.

### BACKGROUND

Mechanical and optical technologies are currently used to create digital scanners for medical imaging and digital printing engines. Typically, imaging apparatus comprising scanning and imaging systems are able to create large images of slide specimens by capturing multiple smaller images of a number of target areas, known as swathes or swathe scans or tiles, and stitching these multiple smaller images together. When capturing the multiple swathes, the scanning stage on which the specimen is held moves to a new imaging position between each swathe or tile so that a new target area of the slide specimen can be imaged. However, the scanning stage must settle, after it has been moved, before each image is captured in order to reduce imaging artefacts caused by imaging a moving target area.

These scanning systems are preferably isolated from external vibrations, for example vibrations due to external environmental factors, as much as possible. Generally, this isolation is implemented in the form of elastic anti-vibration (AV) mounts which may be placed between the scanning system and the rest of the scanning apparatus and, optionally, between the scanning apparatus and the surface on which the scanning apparatus is placed such as a work bench. Typically, the stage of the scanning system will be mounted to a scanner structure, which may comprise the imaging system for the scanning system, and the scanner structure may itself be mounted to mounts, such as AV mounts. As such, when the scanning stage moves from one imaging position to another imaging position, the acceleration and deceleration of the moving scanning stage cause an oscillation of the scanner structure on the mounts. This oscillation causes imaging artefacts unless enough time is left for the oscillations to decay sufficiently before the image is captured.

In order to reduce imaging artefacts, it is known for the scanning system to check, e.g. through a movement sensor or a camera and a controller, that the position of the scanning stage remains within a defined range of its target imaging position, for a defined time before an image of the target area is captured. Whilst this does reduce the likelihood of imaging artefacts, this method slows down the overall image capture rate due to the additional wait time between consecutive image captures.

Another known solution is to use post-processing techniques to try to eliminate some of the effects caused due to oscillation. However, there are limits in the rate of change and magnitude of position error that this technique can compensate for.

### SUMMARY OF INVENTION

According to a first aspect, there is provided a scanning device for scanning a target area of a sample. The scanning device comprises a transportation system arranged to support the sample and move the target area between a first location and a second location, and a casing configured to support the transportation system. The casing is mechanically coupled to the transportation system such that movement of the transportation system induces oscillatory motion in the casing. The scanning device also comprises a controller configured to: i) accelerate the transportation system in a first direction, from the first location to the second location, causing a reaction force in the second direction and initial oscillatory motion of the casing in the second direction; and ii) decelerate the transportation system in the first direction to bring the transportation system to a rest when the sample is in the second location, producing a reaction force on the casing in the first direction which balances oscillatory movement of the casing in the second direction, to bring the casing to rest.

The present invention aims to provide an improved scanning device to be used with any apparatus where a target area of a sample is displaced from a first position to a second position so that the target area has a shorter positional stabilisation time once it reaches the second position. Such scanning device may be conveniently used in, for example but not limited to, an imaging apparatus for scanning and imaging material, for example biological material, generally in the form of slide specimens such as human tissue specimens.

The movement of the transportation system causes an elastic distortion of the casing which is, at least in part, displaced in a direction opposite to the direction in which the transportation system moves, as a vibration of the casing is induced.

Advantageously, the scanning device is arranged such that vibrations induced in the casing during the acceleration of the transportation system are cancelled out during the deceleration of the transportation system. As the transportation system accelerates, its movement causes an acceleration of the casing in the opposite direction. Since the casing is in a fixed position, the acceleration of the transportation system and the reactionary acceleration of the casing causes an elastic deformation of the casing, for example of an elastic portion of the casing and/or of an elastic portion provided to the casing, which is/are thus brought to store a resulting amount of elastic potential energy. In particular, the elastic portion starts oscillating back and forth from its resting position, i.e. the position occupied in the absence of any acceleration or deceleration and/or vibrations. By suitably timing the deceleration of the transportation system to occur as the casing is moving in the opposite direction to the transportation system and towards its resting position, the momentum of the transportation system and casing cancel each other out and the whole scanning device comes to rest, substantially when the transportation system reaches the second position. This shortens the time necessary for the target area to reach a positional stability and so the time needed to collect an image of the target area without artefacts being introduced to the image as a result of the induced vibrations in the overall system.

The scanning device preferably further comprises at least one mount configured to support the casing on an external surface. In some examples, the at least one mount is attached to the casing and arranged to support the casing. Preferably, the scanning device comprises a plurality of mounts, for example two mounts or more. The casing may be provided with at least one mount configured through which the scanning device rests on an external surface.

Preferably the at least one mount comprises elastically deformable material. The at least one mount may be the elastic portion described above. It will be understood that, while the contact surface of the mount and the external surface would not change or move while the transportation system moves, the portion of the mount(s) between the contact surface and the casing would elastically deform oscillating from one side to the other around its resting position.

The one or more mounts can be arranged to isolate the scanning device from the surrounding environment, to help ensure that vibrations present within the external environment are not passed into the scanning device which could affect the scanning process.

In some examples, movement of the casing is arranged to induce vibrations in the at least one mount, the vibrations causing subsequent oscillatory motion of the casing. Generally, when the casing is initially displaced in the opposite direction to the transportation system, due to the movement of the transportation system, the at least one mount elastically distorts. The elastic potential energy stored in the mount acts to accelerate the casing in the same direction to the direction of movement of the transportation system until the casing reaches a maximum displacement, and the casing starts oscillating back and forth on the mount.

This oscillatory motion can be measured, using any suitable technique for determining the oscillatory motion of an object, and an oscillation profile can be determined. This may form part of a calibration process. The oscillation profile can advantageously be used to ensure the timing of the deceleration is chosen so that the momentum of the transportation system and the momentum of the parts of the scanning device oscillating on the mount, including the casing, have substantially the same modulus but are directed in opposite directions so as to substantially cancel each other out and allowing the whole scanning device to come to rest substantially when the transportation system, and in turn the target area, reaches the second position. The biological sample located on the target area can therefore be imaged without artefacts being introduced to the image as a result of the induced vibrations in the scanning device, and the time necessary for the target area to reach a positional stability is significantly reduced.

The at least one mount may have a low damping factor. This may help ensure that the at least one mount does not itself damp the scanning device too significantly so that timed deceleration instead can be used to counter oscillations, rather than relying on the damping provided by the mount. This results in more accurate and more consistent and effective damping during repeated acceleration and deceleration events, for example between consecutive experiments or between consecutive image samples. In this context, the cancellation of momentum may be thought of as effective damping because, from the point of view of image capture, the oscillatory motion has effectively been removed by being cancelled out.

Preferably, the at least one mount comprises a first damping factor in a first direction and a second damping factor in another (second) direction. The first damping factor may be different from the second damping factor. For example, the first damping factor may be greater or smaller than the second damping factor. The first direction may be perpendicular to the other (second) direction. In this way, the mount provides different levels of damping in different directions. For example, the mount may provide a high level of damping in one direction and a low level of damping in another direction. This may give the scanning device different effective sensitivities to motion in different directions. As a result of the different damping factors, vibrations or oscillations induced in the system in one direction may be countered for by the mount more effectively in the first direction compared to the second direction, or vice versa.

In some exemplary apparatus, the first direction may be parallel to the direction of motion of the target area, from the first location to the second location, and the second direction may be perpendicular to the direction of motion of the target area. In this case, preferably, the first damping factor is less than the second damping factor. This has the effect that the mount provides a greater countering effect in the second direction perpendicular to the direction of motion of the target area, and so the mount provides a large amount of damping for oscillatory motion in this direction. Similarly, the mount provides a lesser countering effect in the first direction parallel to the direction of motion of the target area, and so the mount provides a small amount of damping for oscillatory motion in this direction. As discussed above, this ensures that effective damping in the direction parallel to the direction of motion of the target area is provided through timed deceleration rather than by the mount, which helps ensure consistent effective damping.

It will be understood that, while the at least one mount is preferably comprised in the scanning device, and preferably attached to the casing, the presence of at least one mount is not essential, and the same momentum-cancelling effect can be achieved by considering the casing as the feature where elastic potential energy is stored and vibrations are induced and subsequently cancelled.

The casing may also have a low damping factor. The casing may have any one of more of the above damping features described with reference to the at least one mount. The advantages associated with a casing having a low damping factor are substantially the same as described above with reference to the at least one mount.

The scanning device may further comprise a computing system, preferably communicatively coupled to the controller, and wherein the computing system is preferably arranged to instruct the controller to accelerate the transportation system and decelerate the transportation system. In some examples, the controller may comprise a driving system and the computing system may therefore be considered as being in communication with the driving system, for example through the controller. The driving system may be configured to cause movement, for example acceleration and deceleration, of the transportation system via signals or instructions received from the controller and/or the computing system.

The computing system may be configured to detect and monitor the oscillatory motion of the casing. The computing system may be configured to determine an oscillation profile of the casing. The oscillation profile may also be referred to as a moving profile, and the oscillation profile substantially corresponds to the oscillatory motion of the casing. The computing system may be configured to store the oscillation profile of casing in a memory of the computing system. The computing system may be configured to calculate a time at which the controller decelerates the transportation system, so that the calculated time is such that the deceleration occurs at substantially the same time as the casing moves in the opposite direction to the direction of deceleration. Thus, the computing system may be configured to calculate a time at which the controller decelerates the transportation system based, at least in part, on the stored oscillation profile of the casing. In this way, a relationship may be established between the movement of the transportation system and the oscillatory motion of the casing.

The computing system is preferably arranged to instruct the controller to accelerate the transportation system and decelerate the transportation system within one complete oscillation cycle. Preferably, the computing system is arranged to cause the controller to accelerate the transportation system and decelerate the transportation system within the first oscillation cycle. In this way, the momentum of the casing will closely match the momentum of the transportation system, and the target area, after one complete cycle of oscillations because the oscillations will not yet have decayed significantly, and so the cancellation will be most effective at this point.

According to another aspect there is provided an imaging apparatus comprising a scanning device in accordance with any of the examples described above.

According to another aspect, there is provided a method of operating a scanning device comprising the steps of accelerating a transportation system in a first direction to move a sample from a first location to a second location, wherein accelerating the transportation system induces oscillatory motion on a casing that is mechanically coupled to the transportation system, the casing configured to support a scanning system for scanning the sample, wherein the acceleration causes a reaction force in the second direction and initial oscillatory motion of the casing the second direction; and decelerating the transportation system in the first direction to bring the transportation system to a rest when the sample is in the second location, wherein the deceleration causes a reaction force on the casing in the first direction which balances oscillatory movement of the casing in the second direction to bring the casing to rest.

In some examples, the motion of the casing comprises a plurality of oscillation cycles and the decelerating is timed to occur during a first oscillation cycle of the casing. In some other examples, the decelerating is timed to occur during a successive oscillation cycle to the first oscillation cycle of the casing.

Preferably, the casing oscillates at its resonant frequency.

According to another aspect there is provided a method of generating a move profile for a scanning device comprising: providing an impulse to a transportation system causing the transportation system to move a target area from a first location towards a second location, wherein movement of the transportation system causes movement of a casing of a scanning system; measuring the resonant frequency of the casing; calculating at least one of acceleration and / or declaration of the casing based on the resonant frequency; and generating a move profile of the casing based on the calculated acceleration and / or deceleration.

### BRIEF DESCRIPTION OF DRAWINGS

The present invention will now be described by way of example with reference to the accompanying drawings in which:

FIG. 1 shows a schematic scanning device;

FIG. 2 shows part of a schematic scanning device;

FIG. 3 shows a schematic scanning device during a part of a scanning process;

FIG. 4 shows a schematic scanning device during a part of a scanning process;

FIG. 5 shows a schematic scanning device during a part of a scanning process;

FIG. 6 shows a schematic scanning device during a part of a scanning process;

FIG. 7 is a graph showing change in displacement over time;

FIG. 8 is a graph showing change in velocity over time;

FIG. 9 is a graph showing change in acceleration over time; and

FIG. 10 shows exemplary move profile.

### DETAILED DESCRIPTION

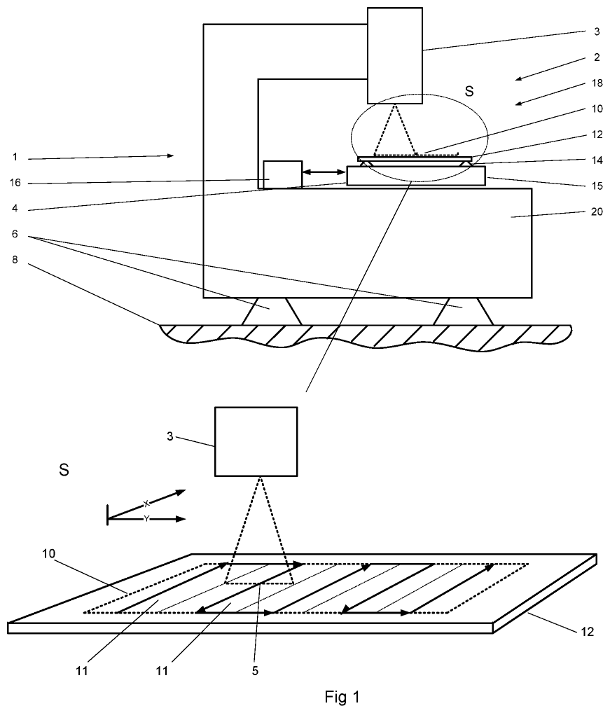

FIG. 1 illustrates an exemplary scanning device 1 for imaging samples, such as biological samples. The scanning device 1 generally comprises a scanning system 2 for scanning the sample. The scanning device 1 comprises an imaging system 3 comprising an imaging collecting device (for example a camera or the like) for collecting images of the sample, and an illumination system to shed light onto the sample. The scanning device 1 further comprises a transportation system 4 for moving the sample relative to the imaging system 3. The transportation system 4 is supported by a casing 20. In some examples, such as that shown in FIG. 1, at least one mount 6 is arranged to support the scanning device 1 on an external surface 8, for example a table, workbench, or the floor.

The sample to be scanned is placed onto a target area 10 of a rigid substrate 12, to form a slide specimen. The substrate 12 is formed from a material that is compatible with the experiment under consideration. The substrate 12 may be sized to receive one or more samples in the target area 10. The target area 10, which may also be referred to as the sample area, is the area, for example, to be scanned by the scanning system 2. The substrate 12 may be a discrete object in the form of a microscope slide, for example based upon a borosilicate glass slide, as these are readily available and suitable for most general applications. However, as will be appreciated, the substrate 12 composition can be selected to be compatible with individual experiments, for example the substrate could be glass or plastic. The substrate 12 can be flat or indented with microwells and structures.

As mentioned, the transportation system 4 moves the target area 10, and therefore moves the sample, relative to the imaging system 3 to image the target area 10.

The transportation system 4 includes a support mechanism 14 onto which the substrate 12 is placed. The support mechanism 14 therefore supports the substrate 12. The transportation system 4 also includes a driving system 16 for moving the support mechanism 14 relative to the imaging system 3. The driving system 16 moves the support mechanism 14 left-to-right in the y-direction as well as back-and-forth (i.e. front-to-back) in the x-direction. This allows the support mechanism 14 to be positioned at different locations relative to the imaging system 3, which has the effect of positioning the substrate 12, and the target area 10, at different locations relative to the imaging system 3. It will be understood that the x-and y-directions are perpendicular directions in a horizontal plane. A controller is coupled to the transportation system 4, via the driving system 16, such that the controller controls the movement of the transportation system 4 via the driving system 16. The controller accelerates the transportation system 4 to move the target area 10 away from the first location towards the second location, and decelerates the transportation system 4 when the target area 10 is approaching the second location.

The scanning system 2 is supported by a casing 20. In some examples, the scanning system 2 is located within the casing 20 and so is supported within the casing 20. In other examples, the scanning system 2 is external to the casing and to the casing 20 provides external support to the scanning system 2.

In some examples, for example that illustrated in FIG. 1, the at least one mount 6 may be attached, directly or indirectly, to the casing 20. The casing 20 may also house other components such as computing components, electronics, and power supply components for at least partially controlling and powering the scanning system 2. A computer control system may be connected to some or all of the individual components of the scanning device 1 including the scanning system 2, transportation system 4, the driving system 16, and all the sub-components of these systems. All the individual components and sub-components of the scanning device 1 are, therefore, computer controlled providing a fully automated, computer-controlled apparatus. A computer program runs on the computer control system which can be programmed by a user. The user is able to input the required parameters and details of the imaging sequence into the computer program so that when the program is run, the scanning device 1 carries out the required imaging sequence without any further interaction from the user, until the imaging sequence has been completed.

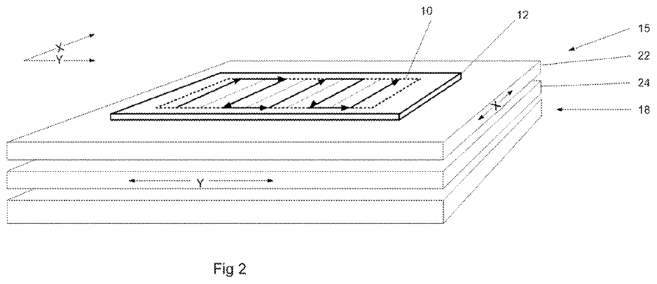

As shown in FIG. 2, the support mechanism 14 is in the form of a stage 15. The substrate 12 is, therefore, positioned on, and supported by, the stage 15. The stage 15 is coupled to the driving system 16 which moves the stage 15. The driving system 16 moves the stage 15 laterally, the movement being confined to a single horizontal plane. The stage 15 can therefore move left and right as well as forwards and backwards. The driving system 16 is, therefore, a multi-directional driving system, for example an X+Y driving system. The driving system 16 ensures that the target area 10 is accurately positioned relative to the imaging system 3, the driving system 16 allowing the position to be finely tuned, if necessary, in the x- and y-directions. As will be appreciated, in some examples the driving system 16 can be a single direction driving system, for example the driving system 16 may move the stage 15 in either the x-direction or the y-direction.

The scanning system 2 may comprise a scanner 18 which scans the target area 10 of the substrate 12. In the example shown, the scanner 18 may take the form of a digital scanner 18.

The digital scanner 18 may be arranged to perform swathe-scanning across the entire target area 10 of the substrate 12, as shown in portion S of FIG. 1. The target area 10 can be thought of as being split up into multiple adjacent sub-sections 11. Swathe-scanning involves scanning one or more sub-sections 11 of the target area 10 sequentially when the target area 10 is larger than the field of view (FOV) 5 of the digital scanner 18. The proportion of the total surface area of the substrate 12 which can be viewed by the digital scanner 18 at one time is determined by the FOV 5 of the digital scanner 18. The FOV of the digital scanner 18, therefore, determines what percentage of the surface area of the target area 10 can be scanned at one time.

Since, in general, the target area 10 onto which the sample is placed will be larger than the FOV 5 of the digital scanner 18, the digital scanner 18 is only able to view a limited proportion of the sample in the target area 10 at a time. In order to scan the entire sample on the target area 10, the sample needs to be moved with respect to the FOV 5 of the digital scanner 18.

The digital scanner 18 detects what proportion of the sample the target area 10 covers so that, when the swathe-scan is performed, the entire target area 10 is captured. The digital scanner 18 is therefore able to ensure that all of the sample corresponding to the target area 10 is scanned. As will be appreciated, different swathe-scans can be combined together using algorithms which identify the edges of different swathe-scans and match up the edges of consecutive swathe-scans to produce a final, large, overall scan of the entire scanning sequence experiment undertaken.

The swathe-scan is a continuous, movement, for example at a speed of approximately 10 mms⁻¹, in one direction within a horizontal plane. For example, for an initial y-coordinate, the swathe-scan is completed in the x-direction to scan the sample for all x-coordinates. Once the swathe-scan has been completed at an initial y-coordinate, the target area 10 needs to be moved in the y-direction before the next swathe in the x-direction is completed, in order to ensure the entire sample area 10 is scanned. The movement of the transportation system 4, and in turn the target area 10 in the y-direction, is an example of what has been described above as the movement of the transportation system 4 from a first position to a second position. This is represented in FIG. 2 by the arrows in the y-direction (or −y-direction), while the movement in the x-direction, described in connection with the swathe-scan is represented in FIG. 2 by the arrows in the x-direction (or −x-direction).

Ideally, between each change in position between swathe-scans, all the parts of the equipment are stabilised so there are no visualisation issues, i.e. the captured scan is not blurry as a result of the movement of the support mechanism 14. In practice, however, this is rarely the case and the resulting scan comprises artefacts as a result of motion of at least some of the components of the scanning device 1. This problem is described in more detail below.

The transportation system 4 can be thought of as being arranged to move the target area 10 between a first location, in which the scanning system 2 scans a first imaging area, and a second location, in which the scanning system 2 scans a second imaging area. Here, each of the first and second imaging areas may correspond to the area scanned by a swathe-scan.

As can be more clearly seen in FIG. 2, the stage 15 comprises a plurality of plates, including a first plate 22 and a second plate 24. The first plate 22 and the second plate 24 are in vertical alignment with each other, such that the first plate 22 and the second plate 24 are positioned one on top of the other. Both the first plate 22 and the second plate 24 are connected to the driving system 16. The first plate 22 holds the substrate 12 and is configured to move the substrate 12 and target area 10, in the x-direction during the swathe-scan. The second plate 24 is configured to move the first plate 22, and therefore the substrate 12 and the target area 10, from the first location to the second location in the y-direction. In other words, the first plate 22 moves during the swathe-scan and the second plate 24 moves between swathe-scans. The first plate 22 is generally lighter in mass than the second plate 24, ensuring that the first plate 22 can move rapidly during the scan.

The casing 20 is mechanically coupled to the transportation system 4 such that movement of the transportation system 4 induces oscillatory motion in the casing 20. In particular, as a result of the additional mass of the second plate 24, when the stage 15 moves from the first location to the second location between swathe-scans movement is induced in the casing 20 in the opposite direction, and so movement of the transportation system 4 causes movement of the casing 20. Since the scanning system 2 is supported by the casing 20, movement of the transportation system 4 therefore causes movement of the scanning system 2. In particular, when the stage 15 moves from the first location to the second location, an elastic distortion is caused in the casing 20 which results in a displacement of at least part of the casing 20 in the opposite direction, and so movement of the stage 15 causes movement of the casing 20 and the scanning system 2.

More particularly, as the stage 15 initially accelerates from the first location towards the second location (i. e acceleration in a first direction), due to the driving system 16 accelerating the second plate 24, a reaction force in the opposite direction (i.e. a second direction) is imparted into the scanner 18 which causes a displacement of the scanner 18 in the second direction, as illustrated in FIG. 3. In the example illustrated, the one or more mounts 6, to which the scanning system 2 is attached, are made of elastically deformable material and so movement of the scanning system 2 results in an elastic distortion of the mounts 6. The elastic potential energy stored in mounts 6 is released, causing acceleration of the scanning system 2 in the same direction as the direction of motion of the stage 15 (i.e. in the first direction), as shown in FIG. 4. If no other forces act on the scanning device 1, the scanning system 2 will continue to oscillate back and forth on the mounts 6, as shown in FIG. 5, before gradually coming to rest. The oscillations of the scanning system 2 are perpendicular to the direction of the swathe-scan. The oscillating scanning system 2 will therefore introduce imaging artefacts if the next swathe-scan has begun before the oscillations decay sufficiently to a level at which they will not negatively affect image capture. These oscillations need to be compensated for to ensure that imaging artefacts are not introduced. As has been discussed, although elastic distortion has been described with reference to the mounts 6, it will be appreciated that the casing 20 will also elastically distort due to movement of the stage 15. As will be appreciated, in scanning devices which do not comprise mounts, the movement of the stage 15 results in elastic distortion of the casing 20 only.

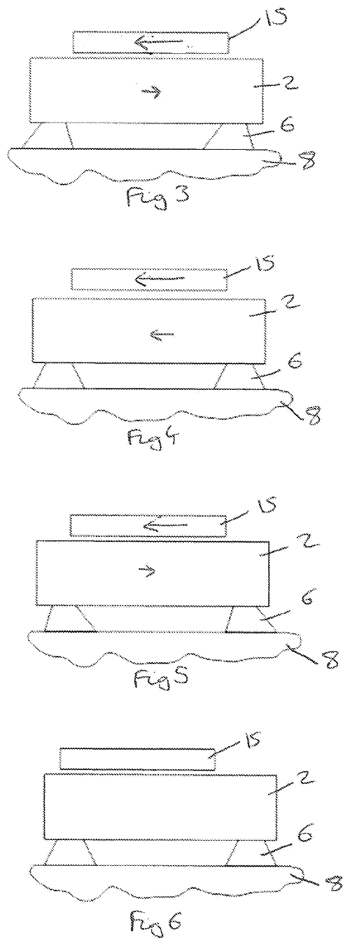

It has been found that by timing the deceleration of the stage 15, as the stage 15 reaches the second location, to occur at substantially the same time as the scanning system 2 is moving in the opposite direction to the stage 15, towards its resting position, having completed ¾ of the first or a successive oscillation cycle, the momentum of the stage 15 and the momentum of the scanning system 2 substantially cancel each other out so that the scanning device 1 comes to rest, as shown in FIG. 6, allowing subsequent scan capture to take place in a stable system. The transportation system 4 is decelerated in the first direction (or accelerated in the second direction, corresponding to negative acceleration i.e. declaration in the first direction) to bring the transportation system 4 to a rest when the sample is in the second location. The deceleration imparts a reaction force on the casing 20 in the first direction (i.e. in the direction which is opposite to the force being applied to the transportation system 4 to slow the transportation system 4 down), this reaction force balancing the oscillatory movement of the casing 20 in the second direction and therefore bringing the casing 20 to rest. The oscillation of the scanning system 2 is effectively minimised by timing the stage 15 deceleration to occur when the displacement of the scanning system 2 is the same magnitude and in the opposite direction as during the acceleration of the stage 15.

The controller connected to the driving system 16 is configured to accelerate the transportation system 4, in particular the second plate 24 of the stage 15, to move the target area 10 away from the first location towards the second location and decelerate the transportation system 4, in particular the second plate 24 of the stage 15, when the target area 10 is approaching the second location until the target area 10 is stationary in the second location. The driving system 16 decelerates the transportation system 4, in particular the second plate 24 of the stage 15, at substantially the same time as the scanning system 2 moves in the opposite direction to the direction of deceleration. This means that the controller is configured to time the deceleration of the transportation system 4 to occur as described above. The timing of the deceleration is such that as the second plate 24 of the stage 15 is about to stop moving once it reaches the second location, the scanning system 2 which is moving in the opposite direction has reached its maximum displacement so that the combined momentum of the moving second plate 24 and the oppositely moving scanning system 2 cancel out and both the second plate 24 and the scanning system 2 come to rest. In effect, the second plate 24 of the stage 15 is timed to move in accordance with the oscillation frequency of the scanning system 2. This then means that the forces imparted into the casing 20 by the transportation system 4 cancel out the forces present in the transportation system 4 as a result of the acceleration and deceleration. The initial acceleration of the transportation system 4 imparts an initial force into the casing 20, initiating the oscillatory motion. This initial force is in the opposite direction to the direction of the acceleration, and causes the casing to initially displace in the opposite direction to the direction of the acceleration. The deceleration of the transportation system 4 imparts a subsequent force into the casing 20, to minimise and preferably substantially cancel the oscillatory motion. This subsequent force is in the opposite direction to the direction of the initial force and so acts to counter the forces within the oscillating casing 20 which is travelling in the second direction, thus causing the casing 20 to stop moving when the transportation system 4 stops moving at the second location.

Preferably, the casing 20 and/or mounts 6 have a relatively low damping factor, for example a damping ratio <0.1, in order that the oscillations are substantially corrected for by relying on the cancellation of momentum present within the scanning device 1 rather than through damping by the mounts 6. In this way, the mounts 6 function to isolate the scanning device 1 from external vibrations, such as those present in the external environment, rather than from vibrations originating and present within the scanning device 1. Mounts 6 having a higher damping factor can more effectively isolate the scanning device 1 from external vibrations.

In some exemplary scanning device 1, the mounts 6 may have a damping factor that is different in the x- and y-directions. For example, the damping factor in the direction perpendicular to the direction of the swathe-scan (y-direction) may be less than the damping factor in the direction parallel to the direction of the swathe-scan (x-direction). Said another way, the damping factor in a direction that is parallel to the direction of movement from the first location to the second location may be less than the damping factor in a direction that is perpendicular to the direction of movement from the first location to the second location. Of course, in some exemplary scanning device 1, the mounts 6 may have a damping factor that is the same in both the x- and the y-directions.

The moving profile of the scanning device 1, which describes how the scanning device 1 moves as a result of the elastic deformation of the mounts 6, and subsequent oscillation of the scanning system 2, can be calibrated initially in order to determine the oscillation profile, which can subsequently be used to determine the timing of the deceleration for that given scanning device 1. As will be appreciated, the optimum timing will vary between different instances of the scanning device 1 (for example due to imperfections or discrepancies during the manufacturing process), the installation location, and the particular damping properties of the mounts 6.

Preferably, the mounts 6 are chosen to have a damping factor such that the stiffness of the mounts 6 and the mass of the scanning system 2 results in the mounts 6 imparting a resonant frequency into the overall system that is close to the frequency of the movements between successive swathes. In other words, the resonant frequency is similar to the frequency of the movements of the transportation system 4 the first location to the second location.

By having mounts 6 with a low damping factor, the momentum of the scanning system 2 will closely match the momentum of the transportation system 4 at the end of one complete cycle of oscillations on the mounts 6, because the oscillations have not decayed significantly after a single cycle. Given this, it is preferable to initially accelerate and then decelerate the transportation system 4 within the first oscillation cycle of the mounts 6 in order to minimise the amount of energy lost due to the decay of the oscillations and ensure that the momentum of the scanning system 2 substantially matches the momentum of the transportation system 4. In other words, both the acceleration to move the target area 10 away from the first location towards the second location and the deceleration to bring the target area 10 to rest when the target area 10 is in the second location occur within the same oscillation cycle.

Although the discussion regarding damping has referred to the mounts, it will be appreciated that the discussion applies equally to the casing. Thus, when the mounts are not present, it is the casing which has the low damping factor to ensure that the momentum of the scanning system will closely match the momentum of the stage.

As will be appreciated, the deceleration can occur during any oscillation cycle subsequent to the first oscillation cycle. However, some energy will be lost during each subsequent cycle, for example due to friction, and so the cancellation of momentum between the scanning system 2 and the transportation 4 becomes less effective the later the oscillation cycle in which the deceleration occurs.

It should be noted that increasing the stiffness of the mounts 6 or reducing the mass of the scanning system 2 would increase the resonant frequency of the mount 6 and allow for more rapid movement between adjacent swathe-scans.

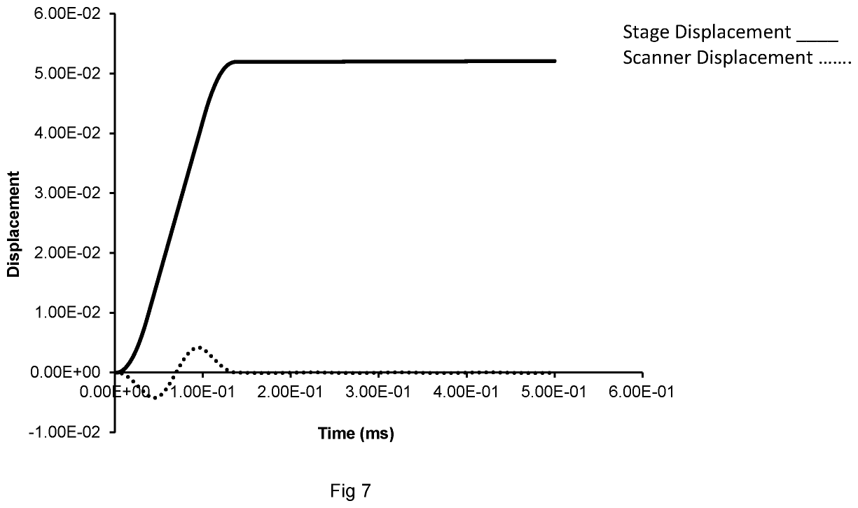

FIGS. 7-9 illustrate some example plots of the changes in displacement, velocity, and acceleration between each motion phase. These simplified plots generally show the stage 15 moving with constant acceleration, within a specific motion phase, that changes substantially instantaneously from one motion phase to the next. In practice, however, the acceleration of the scanning system 2 will change continuously as the mounts 6 are distorted and provide a restoring force which brings the overall system back to equilibrium when stationary.

Turning to FIG. 7 first, this graph shows how the displacement of the stage 15 and the scanning system 2 changes with time. The stage 15 moves from the first location to the second location at substantially a constant speed throughout. The phases of acceleration and deceleration at the first and second locations can be seen on the graph. The scanning system 2 is shown as moving from its initial position to a maximum displacement in the opposite direction to that of the stage 15, oscillating to a maximum displacement in the same direction as that of the moving stage 15, and then back to its initial starting position. The scanning system 2 reaches its maximum negative displacement substantially halfway through the period of time taken for the stage 15 to move from the first location to the second location.

Preferably, the scanning system 2 completes ¾ of an oscillation cycle, before the deceleration imparted to the stage 15 produces a force and momentum which counterbalances the force and momentum experienced by the scanning system 2 so that the scanning system 2 comes to a rest in the initial position. If no deceleration was imparted to the stage 15, then no further force would be imparted to the scanning system and the scanning system 2 would further move from the initial position towards the second location of the stage 15 and back to the initial position, so completing an oscillation cycle and starting a new one until the oscillations progressively faded away while the potential energy is dissipated.

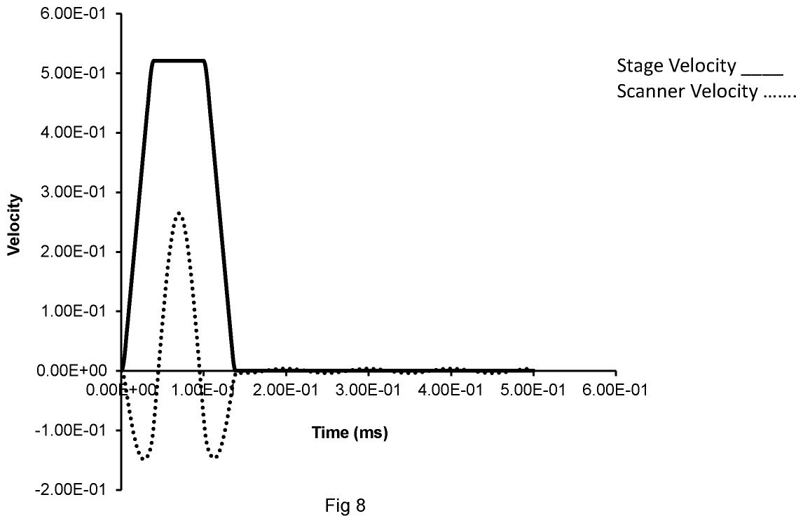

Looking now at FIG. 8, the rate of change of velocity (i.e. the acceleration) of both the stage 15 and the scanning system 2 is illustrated. It is clear from this Figure that the stage 15 initially accelerates (away from the first location), then it travels at a substantially constant speed (between the first location and the second location), and then decelerates (while approaching the second location). During the acceleration phase of the stage 15 in a first direction, the scanning system 2 moves in a second direction opposite to the stage 15 as a result of the reaction force imparted by the stage 15 in the scanning system 2. The imparted force acts to accelerate the scanning system 2 from rest in the second direction which is opposite to the direction of acceleration of the stage 15. During the constant velocity phase of the stage 15, no further force is imparted by the stage 15 to the scanning system 2. Thus, during this phase, the elastic energy from the initial force which has been stored (either in the casing or the mounts if present) is released as a force causing the scanning system 2 to accelerate back in the first direction (and so the scanning system 2 is now moving in the opposite direction to its initial direction of travel). In order to decelerate the stage 15 a force is effectively applied to the stage 15 to slow it down, the force needing to be applied opposite to the direction of travel of the stage 15. This force can be thought of as applying an acceleration to the stage 15 in the second direction. This imparts a corresponding opposite force in the scanning system 2, this corresponding opposite force being applied in the first direction. Since, at this point, the scanning system 2 is moving in the second direction the corresponding opposite force acts against the movement of the scanning system 2, bringing the scanning system 2 to a stop.

Thus, by the time the stage 15 has reached the second location and come to rest, the oscillations of the scanning system 2 have been countered so that the scanning system is also at rest. Both the scanning system 2 and the stage 15 are at rest.

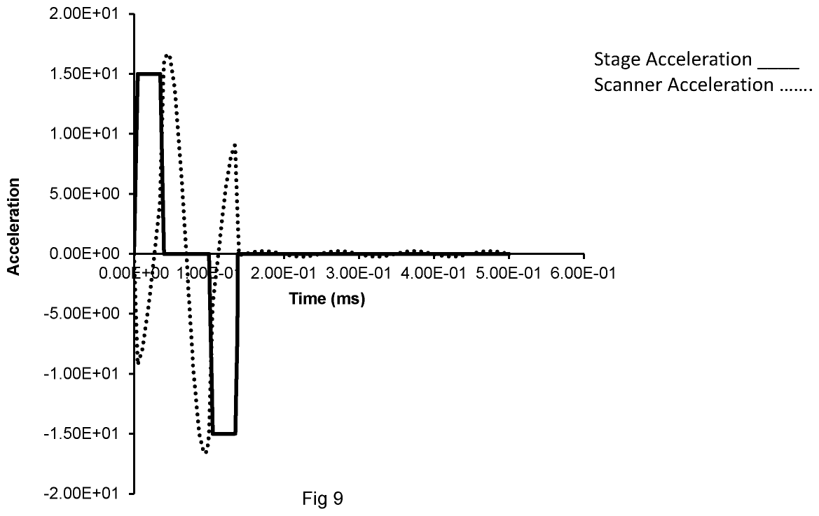

Finally turning to FIG. 9, the rate of change of acceleration of the stage 15 and scanning system 2 is illustrated. As can be seen, the stage 15 initially accelerates, followed by a period of constant velocity, i.e. no acceleration, and then decelerates. The scanning system 2 can also be seen to oscillate between acceleration and deceleration. In particular, as shown in FIG. 9 during the first phase, both the stage 15 and the scanning system 2 begin accelerating at substantially the same time, but in opposite directions. Similarly, during the last phase, both the stage 15 and the scanning system 2 begin decelerating at substantially the same time but in opposite direction.

In summary, the resonant frequency of the mounts 6 is measured and the moving profile of the stage 15 is timed such that certain phases of the movement between the first and second locations coincide with particularly advantageous points within the oscillation cycle. The overall system allows for reduced overall scanning time because the time between adjacent swathe-scans is reduced.

It will be appreciated that the timing and modulus of the deceleration will depend at least on the effective mass of the casing 20 and scanning system 2 and any other component provided thereto which vibrates with the casing 20 and the scanning system 2, the modulus of the acceleration, and the elastic properties of the casing 20 and/or the at least one mount 6.

The function of the mounts 6 is to store elastic potential energy when the mounts are deformed in response to the displacement of the transportation system 4. However, it should be noted that the mounts 6 are a specific implementation of an elastic portion feature. In some implementations, the casing itself may be elastically deformable, or comprise an elastically deformable portion, and carry out the same function of storing elastic potential energy. In some examples, the elastically deformable portion may be a discrete portion of the casing support structure 20 (e.g. AV mounts) or the elastically deformable portion may be the whole casing 20.

Preferably, the deceleration of the transportation system 4 is timed during the fourth quarter of a complete oscillation of the scanning system 2, i.e. when the oscillating structure (which could be the mounts or more generally a casing with an elastic portion) moves towards its resting position.

Although the scanning device has been described with reference to swathe-scans, it will be appreciated that this is not limiting and any way of moving a target area from a first imaging location to a second imaging location known in the art may be used.

As mentioned above, the moving profile which describes how the scanning device 1 moves as a result of the elastic deformation of the casing 20 (and mount 6 when present), and subsequent oscillation of the scanning system 2, can be calibrated initially in order to determine the oscillation profile, which can subsequently be used to determine the timing of the deceleration for that given scanning device 1. The purpose of the calibration is to determine the move profile that minimises the scanner motion after the stage stops moving.

A number of exemplary suitable calibration processes will now be described.

A first method involves testing multiple different move profiles, for example predetermined move profiles or preset move profiles, in order to determine the optimum move profile for the given equipment, the given setup, and the given external environment (e.g., surface on which the equipment is located).

To start the calibration process, the stage is moved using a defined move profile consisting of acceleration, constant velocity, and deceleration phase. The defined move profile can be predetermined and stored on a computing device or based on a computer model. The stage is moved multiple different times, and each time the stage moves the constant velocity at which the stage moves is incrementally increased and the magnitude of motion of the scanner is measured after each move. The effect of the increasing constant velocity of the stage is that the acceleration and deceleration time increases because it takes more time to bring the stage up to speed and bring the stage to rest. A series of data points can be collected and analysed to determine which of the predefined move profiles results in the minimum motion of the scanner after the stage stops moving.

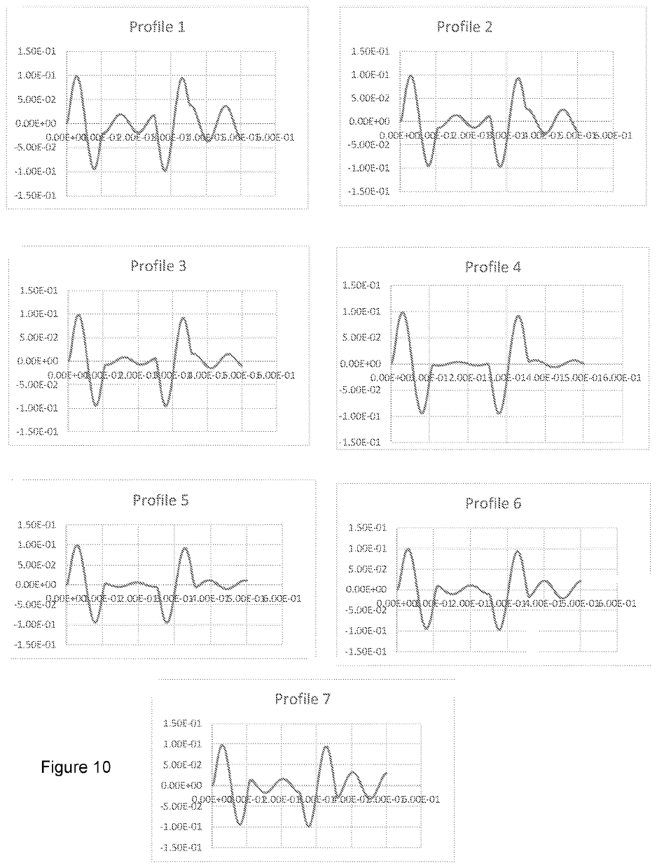

FIG. 10 shows an exemplary series of seven move profiles, each profiles having an increasing constant velocity. As can be seen from FIG. 10, Profile 4 gives the minimum motion magnitude after the stage stops moving. The acceleration and constant velocity from this move profile are selected for the move profiles in the system. If the optimum move profile is between tested values the optimum can be determined by interpolation. The move time could also be incremented by decrementing the acceleration. The error can be measured in the constant velocity phase of the move or at the end of the move. Minimising the error in the constant velocity phase of the move has the advantage that the calibration will be independent of the relative timing of the acceleration and deceleration phases. This allows for moves of varying distance using the same acceleration and velocity. Minimising the error in the stationary phase at the end of the loop has the advantage that the move time can be shorter.

A second calibration method involves measuring the natural resonant frequency of the system by providing an impulse and calculating the optimum move profile based on the resonant frequency. The natural resonant frequency can be measured by providing an impulse to the system and measuring the frequency of the response. This can be done by accelerating the stage for a period which is a small fraction of the resonant period of the scanner on its AV mounts. The frequency of the motion after the impulse is then measured. A move profile can then be defined, where the acceleration and deceleration period are calculated from the measured natural resonant frequency.

A third calibration method involves measuring the natural resonant frequency of the system by providing a swept frequency modulation to the position of the stage, detecting the frequency of peak disturbance, and calculating the optimum move profile.

In order to determine the motion of the scanner, as a result of the movement of the stage, various different sensing method can be employed. For example, a stage encoder can be used to detect errors which occur because motion of the scanner will result in velocity errors in the constant velocity phase of the move and position errors when the stage is stationary after the move. Another option for detecting motion of the scanner is to use an accelerometer on the scanner. A motion sensor can also be used to detect relative motion between the scanner and the case and detect relative motion between the stage and the scanner.

1. A scanning device for scanning a target area of a sample, the scanning device comprising:
a transportation system arranged to support the sample and move the target area of the sample between a first location and a second location;
a casing configured to support the transportation system, the casing mechanically coupled to the transportation system such that movement of the transportation system induces oscillatory motion in the casing; and
a controller configured to:
accelerate the transportation system in a first direction, from the first location to the second location, causing a reaction force in a second direction and initial oscillatory motion of the casing in the second direction; and
decelerate the transportation system in the first direction to bring the transportation system to a rest when the sample is in the second location, producing a reaction force on the casing in the first direction which balances oscillatory movement of the casing in the second direction, to bring the casing to rest.

2. The scanning device according to claim 1, further comprising at least one mount configured to support the casing on an external surface.

3. The scanning device according to claim 2, wherein the at least one mount comprises elastically deformable material.

4. The scanning device according to claim 2, wherein movement of the casing is arranged to induce vibrations in the at least one mount, the vibrations causing subsequent oscillatory motion of the casing.

5. The scanning device according to claim 2, wherein the casing has a low damping factor.

6. The scanning device according to claim 2, wherein the at least one mount has a low damping factor.

7. The scanning device according to claim 6, wherein the at least one mount comprises a first damping factor in the first direction and a second damping factor in the second direction, wherein the first damping factor is different from the second damping factor, and wherein the first direction is perpendicular to the second direction.

8. The scanning device according to claim 7, wherein the first direction is parallel to a direction of motion of the target area and the second direction is perpendicular to the direction of motion of the target area, and wherein the first damping factor is less than the second damping factor.

9. The scanning device according to claim 4, further comprising a computing system, the computing system configured to detect and monitor the oscillatory motion of the casing.

10. The scanning device according to claim 9, wherein the computing system is communicatively coupled to the controller, and wherein the computing system is arranged to cause the controller to accelerate the transportation system and decelerate the transportation system within one complete oscillation cycle.

11. The scanning device according to claim 10, wherein the computing system is arranged to cause the controller to accelerate the transportation system and decelerate the transportation system within a first oscillation cycle.

12. The scanning device according to claim 1, further comprising an imaging system configured to image the target area.

13. An imaging apparatus comprising a scanning device according to claim 12.

14. A method of operating a scanning device comprising:
accelerating a transportation system in a first direction to move a target area of a sample from a first location to a second location, wherein accelerating the transportation system induces oscillatory motion on a casing that is mechanically coupled to the transportation system, the casing configured to support the transportation system,
wherein the acceleration causes a reaction force in a second direction and initial oscillatory motion of the casing the second direction,
decelerating the transportation system in the first direction to bring the transportation system to a rest when the sample is in the second location, and
wherein the deceleration causes a reaction force on the casing in the first direction which balances oscillatory movement of the casing in the second direction to bring the casing to rest.

15. The method according to claim 14, wherein the oscillatory motion of the casing comprises a plurality of oscillation cycles and the decelerating is timed to occur during a first oscillation cycle of the casing.

16. The method according to claim 14, wherein the casing oscillates at its resonant frequency.

17. A method of generating a move profile for a scanning device comprising:
providing an impulse to a transportation system causing the transportation system to move a target area from a first location towards a second location, wherein movement of the transportation system causes movement of a casing of a scanning system;
measuring a resonant frequency of the casing;
calculating at least one of acceleration or declaration of the casing based on the resonant frequency; and
generating the move profile of the casing based on the at least one of acceleration or deceleration.
